(19) United States
(12) Patent Application Publication
(10) Pub. No.: US 2023/0141303 A1
(43) Pub. Date: May 11, 2023

(54) BNCT TREATMENT SYSTEM

(71) Applicant: FUKUSHIMA SiC APPLIED ENGINEERING INC., FUTABA-GUN, FUKUSHIMA (JP)

(72) Inventor: Yuji KOKUBO, FUKUSHIMA (JP)

(21) Appl. No.: 17/910,480

(22) PCT Filed: Feb. 26, 2021
(86) PCT No.: PCT/JP2021/007331

§ 371 Date: Sep. 9, 2022

Related U.S. Application Data
(60) Provisional application No. 62/989,142, filed on Mar. 13, 2020.

(51) Int. Cl. A61N 5/10

(52) U.S. Cl. CPC A61N 5/1071; A61N 5/1077; A61N 2005/109

(57) ABSTRACT

To provide a BNCT treatment system capable of formulating a neutron irradiation mode based on diagnostic data on a subject to be treated. A BNCT treatment system 2, used for performing neutron capture therapy, includes: a Hexatron 3 including first to sixth neutron irradiation devices 3A to 3F which emit neutrons; and a controller 4 configured to control neutron irradiation by the first to sixth neutron irradiation devices 3A to 3F. The BNCT treatment system 2 includes: an HOP 5 configured to formulate a treatment plan (a mode for controlling neutron irradiation of the first to sixth neutron irradiation devices 3A to 3F by the controller 4) based on diagnostic data on a patient PA; an HSP 6 configured to monitor each component member; and a management unit 7 configured to manage the entire system.

### TECHNICAL FIELD

The present invention relates to a BNCT treatment system.

### BACKGROUND ART

Boron neutron capture therapy (BNCT) is known (see, for example, Patent Literature 1).

In Patent Literature 1, an accelerator neutron source and a moderator which moderates neutrons generated by the accelerator neutron source are provided. A boron drug is administered to a patient and an affected area of the patient is irradiated with neutrons moderated by the moderator, so that compound biological effectiveness (CBE) becomes 4 or more by an absorbed dose of the affected part of the patient.

### CITATION LIST

### Patent Literature

Patent Literature 1: Japanese Patent Laid-Open No. 2019-216872

### SUMMARY OF INVENTION

### Technical Problem

In Patent Literature 1, the configuration to increase the compound biological effectiveness is adopted. However, a mode of neutron irradiation, which defines, for example, a target part of a patient to be irradiated with neutrons, is determined by physicists and doctors, which causes a low accuracy of irradiation.

The present invention has been made in view of such background circumstances, and an object of the present invention is to provide a BNCT treatment system capable of formulating a neutron irradiation mode based on diagnostic data on a subject to be treated.

### Solution to Problem

[1] The BNCT treatment system of the present invention is a BNCT treatment system which performs neutron capture therapy using a plurality of neutron irradiation devices which emit neutrons. The BNCT treatment system comprises: a neutron irradiation control unit configured to control neutron irradiation by the neutron irradiation devices; and a neutron irradiation control formulation unit configured to formulate a mode for controlling the neutron irradiation of the neutron irradiation devices by the neutron irradiation control unit, based on diagnostic data on a treatment target.

According to the BNCT treatment system of the present invention, the mode for controlling the neutron irradiation of the neutron irradiation devices by the neutron irradiation control unit can be formulated based on diagnostic data on a treatment target. Accordingly, it is possible to formulate a stable and high-accuracy mode for controlling the neutron irradiation which is free from variations attributed to individual skill levels, as compared with the mode for controlling the neutron irradiation determined by physicists or doctors.

[2] The BNCT treatment system may preferably comprise a verification device configured to calculate treatment dose distribution obtained when the neutron irradiation devices are controlled based on the mode for controlling the neutron irradiation formulated by the neutron irradiation control formulation unit, and verify the mode for controlling the neutron irradiation formulated based on the calculated treatment dose distribution.

According to the above configuration, it is possible to verify the formulated mode for controlling the neutron irradiation before the subject to be treated is actually irradiated with neutrons.

[3] The BNCT treatment system may preferably comprise a monitoring device configured to monitor the neutron irradiation devices.

The above configuration allows the neutron irradiation devices to be monitored for correct operation.

### BRIEF DESCRIPTION OF DRAWINGS

FIG. 1 is a schematic diagram showing a BNCT treatment system.

FIG. 2 shows first to sixth neutron irradiation devices.

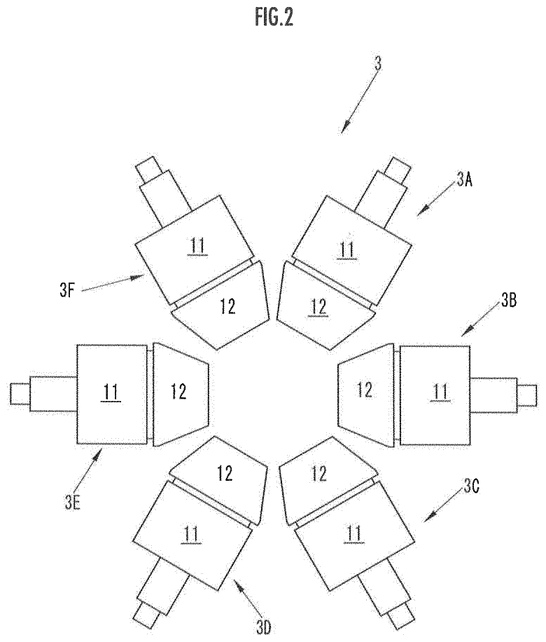

FIG. 3 is a schematic side view showing a neutron irradiation device, a base, and a mobile table.

FIG. 4 shows data on systemic distribution of absorbed dose at the time of biomarker administration.

FIG. 5 shows the systemic data on a patient.

FIG. 6 shows systemic data on the patient with neutron irradiation range being input.

FIG. 7 shows an irradiation sequence.

FIG. 8 shows dose distribution in an X-Y plane in the neutron irradiation range.

FIG. 9 shows the dose distribution in the X-Y plane superimposed on contour information on a patient PA.

Hereinafter, an embodiment of the present invention will be described with reference to the drawings. The embodiment described below is merely exemplary, and the present invention can be applied to embodiments other than the embodiment described below.

In all the drawings used to describe the embodiment, those having identical functions are designated by identical signs to omit repeated description.

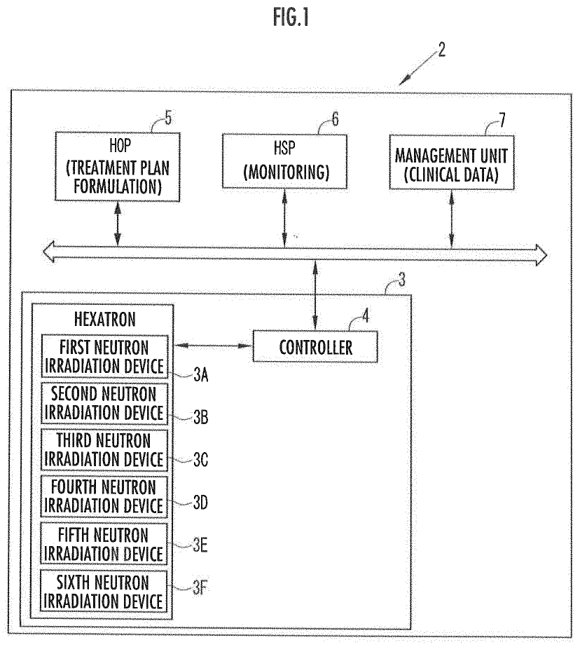

FIG. 1 shows an example of a BNCT treatment system 2 of an embodiment. Hereinafter, the case where the boron drug is BPA will be described as an example. Here, BPA is a compound containing ¹⁰B. In the following description, for the convenience of description, a portion containing cancer cells, in an affected area of the patient, is referred to as a tumor part or simply a tumor, and a portion not containing cancer cells is referred to as normal tissues.

The BNCT treatment system 2, which is used for neutron capture therapy, comprises a Hexatron 3 comprising first to sixth neutron irradiation devices 3A to 3F that emit neutrons, and a controller 4 that controls neutron irradiation by the first to sixth neutron irradiation devices 3A to 3F. The Hexatron 3 (first to sixth neutron irradiation devices 3A to 3F) and the controller 4 are disposed and used in hospitals, for example.

The BNCT treatment system 2 also comprises a HexaVision Oncology Panel (HOP) 5 which formulates a treatment plan (a mode for controlling the neutron irradiation of the first to sixth neutron irradiation devices 3A to 3F by the controller 4) based on the diagnostic data on a patient PA (subject to be treated), a HexaVision SCADA Panel (HSP) 6 which monitors each unit, and a management unit 7 which manages the entire system. The HOP 5, the HSP 6, and the management unit 7 are installed and used, for example, in a company which manufactures and sells the Hexatron 3 (first to sixth neutron irradiation devices 3A to 3F). Each unit may be connected by an internal LAN, for example.

Since the first to sixth neutron irradiation devices 3A to 3F are identical in structure, description is given by taking the first neutron irradiation device 3A as an example.

The first neutron irradiation device 3A comprises an accelerator neutron source 11 and a moderator 12. In an example, the moderator 12 is constituted by including aluminum fluoride (AlF₃) with a thickness of 10 [cm] to 20 [cm].

The accelerator neutron source 11 generates neutrons. In an example, the accelerator neutron source 11 is constituted by including an electrostatic accelerator. Here, the accelerator neutron source 11 has a neutron source strength of, for example, about 4.0×10¹⁰ to 8.0×10¹⁰[neutrons/sec].

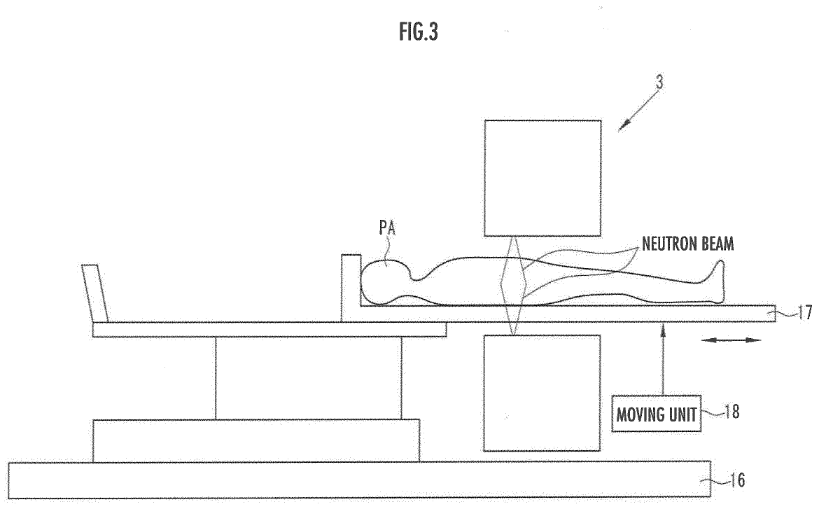

The moderator 12 moderates the neutrons generated by the accelerator neutron source 11 to an energy level optimum for treatment. A tumor of the patient PA is irradiated with the neutrons moderated by the moderator 12. In the example shown in FIG. 3, the patient PA has a tumor in the abdomen, for example, and the tumor of the patient PA is irradiated with neutrons from a prescribed region on the skin surface of the abdomen of the patient PA.

In the BNCT, when the patient PA is administered BPA (boron drug), formed by combining boron with a drug having a characteristic of staying in a tumor part irradiated with neutrons, by intravenous drip or the like, the tumor part of the patient PA incorporates the administered BPA. When the tumor part incorporating BPA is irradiated with neutrons (thermal neutrons), radiation (for example, alpha rays, ⁷Li, etc.) is generated inside the tumor due to nuclear reaction between boron and neutrons. The generated radiation damages the tumor part. As a result, the BNCT destroys the tumor part with high selectivity.

When the affected area of the patient PA is irradiated with neutron beams from the first to sixth neutron irradiation devices 3A to 3F, the neutrons cause nuclear reaction with hydrogen nuclei and nitrogen nuclei included in the affected area and with boron nuclei in the BPA. As a result, high-energy particle rays, such as proton rays, carbon nucleus rays, alpha rays, and lithium nucleus rays, resulting from the nuclear reaction energize affected tissues. In this case, the affected area absorbs energy of some gamma rays, among the gamma rays generated by neutron irradiation from the first to sixth neutron irradiation devices 3A to 3F.

The Hexatron 3 (first to sixth neutron irradiation devices 3A to 3F) is disposed in an irradiation chamber provided in a hospital. The irradiation chamber includes a base 16, a mobile table 17 attached to the base 16 so as to be movable in a horizontal direction, and a moving unit 18 which moves the mobile table 17.

The mobile table 17 is provided such that a portion on which the patient PA is disposed is movable in the horizontal direction (right-left direction in FIG. 3), the portion being surrounded with the first to sixth neutron irradiation devices 3A to 3F. The mobile table 17 is provided so as to allow neutron rays to pass through. The controller 4 controls driving of the moving unit 18.

The HOP 5 is configured to formulate a treatment plan (a mode for controlling the neutron irradiation of the first to sixth neutron irradiation devices 3A to 3F by the controller 4) based on the diagnostic data on the patient PA. The HOP 5 loads a treatment plan formulating program stored in a memory (not shown) to formulate a treatment plan.

The HSP 6 monitors the Hexatron 3 (first to sixth neutron irradiation devices 3A to 3F), the controller 4, the HOP 5, etc.

The management unit 7 monitors a well-known radiation area monitor, an access management system, a cooling system, a gas and vacuum system, etc. (none of which are shown), in addition to the Hexatron 3 (first to sixth neutron irradiation devices 3A to 3F), the controller 4, the HOP 5, and the HSP 6. The management unit 7 also stores various clinical data for use in formulating a treatment plan with the HOP 5. The clinical data are updated as needed.

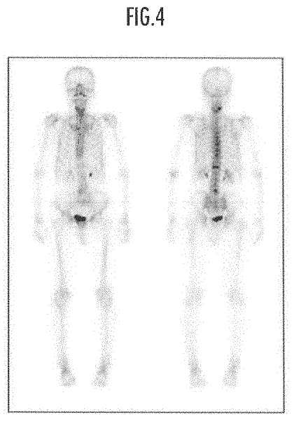

The hospital which treats the patient PA acquires data on systemic distribution of absorbed dose of the patient PA (planar image (DICOM)) when the patient PA is administered a biomarker (see FIG. 4). The hospital also acquires a CT image and an MRI image of the patient PA. The hospital then transmits the planar image (DICOM), the CT image, and the MRI image to the HOP 5 as diagnostic data on the patient PA. Note that at least one of the CT image and the MRI image may be transmitted.

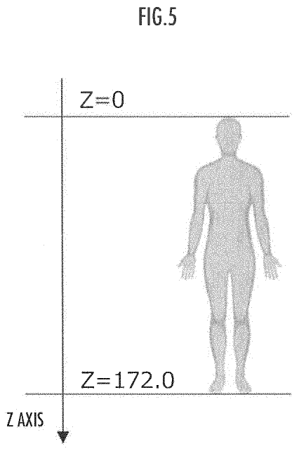

As shown in FIG. 5, the HOP 5 generates systemic data on the patient PA based on the planar image (DICOM) of the patient PA, and displays the data on a display unit (not shown). In the systemic data on the patient PA, a Z axis is generated with a head top part being zero. For example, when the patient PA is 172 cm tall, a sole part is Z=172 cm with the head top part being Z=zero.

The HOP 5 generates a neutron irradiation sequence as the treatment plan, based on the planar image (DICOM) at the time of biomarker administration, the CT image, and the MRI image.

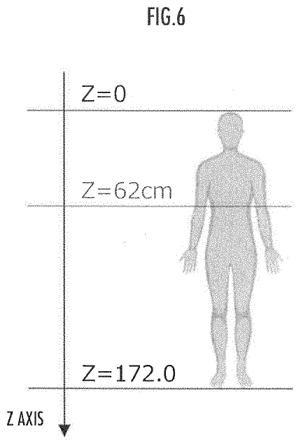

In generation of the neutron irradiation sequence, as shown in FIG. 6, the HOP 5 determines a neutron irradiation range (a prescribed range around Z=62 cm in the present embodiment) based on the planar image (DICOM) indicating the systemic distribution of absorbed dose at the time of biomarker administration, the CT image, and the MRI image. For example, in the planar image (DICOM) at the time of biomarker administration, a portion where the biomarker has a high absorbed dose is set as the neutron irradiation range (the prescribed range around (Z=62 cm) on the Z axis. An administrator of the HOP 5 and a doctor in charge of the patient PA may discuss and determine the neutron irradiation range based on the planar image (DICOM) at the time of biomarker administration, the CT image, and the MRI image.

Here, when the patient PA is administered BPA (boron drug), formed by combining boron with a drug having a characteristic of staying in the tumor part of the patient PA, the concentration of boron in the tumor part increases. Since the adsorbed dose of the biomarker is proportional to the concentration of boron, a portion with a higher absorbed dose of the biomarker is the tumor part higher in concentration of boron.

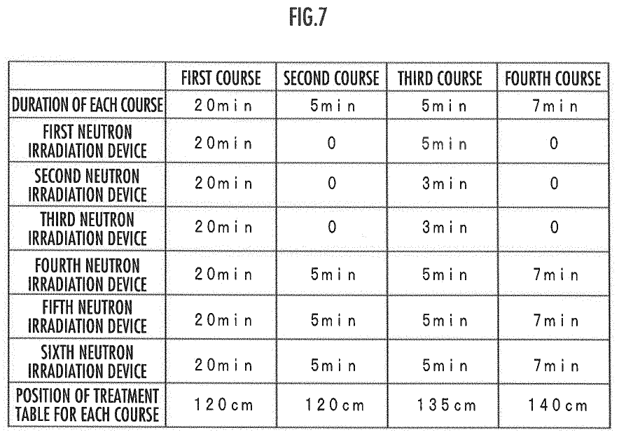

Next, the HOP 5 identifies the tumor part in the neutron irradiation range (prescribed range around Z=62 cm) based on the planar image (DICOM) at the time of biomarker administration, the CT image, and the MRI image. Then, based on the information on the identified tumor (such as size), the HOP 5 refers to various clinical data stored in the management unit 7 to acquire a mode for controlling the neutron irradiation (see FIG. 7) of the first to sixth neutron irradiation devices 3A to 3F, which has actually been executed in a case or cases having similar information on the tumor part (such as size).

The mode for controlling the neutron irradiation of the first to sixth neutron irradiation devices 3A to 3F shown in FIG. 7 is to perform neutron irradiation four times (four courses). The mode for controlling the neutron irradiation includes irradiation time of the first to sixth neutron irradiation devices 3A to 3F in each course. There is an interval of a prescribed time between each of the courses.

Thus, the HOP 5 acquires, as a neutron irradiation sequence as the treatment plan, the center of the neutron radiation range of the patient PA on the Z axis (Z=62 cm) and the mode for controlling the neutron irradiation of the first to sixth neutron irradiation devices 3A to 3F.

Next, the HOP 5 confirms the validity of the neutron irradiation sequence formulated as described above as a treatment plan.

First, based on the planar image (DICOM) indicating the systemic distribution of absorbed dose at the time of biomarker administration, the HOP 5 generates dose distribution in the X-Y plane when neutron irradiation is performed as an irradiation sequence in the mode for controlling the neutron irradiation of the first to sixth neutron irradiation devices 3A to 3F in the neutron irradiation range (prescribed range around Z=62 cm).

In this case, the dose distribution in the X-Y plane, in the case where a portion with a higher absorbed dose of the biomarker is irradiated with neutrons, is generated with use of the fact that a portion with a higher absorbed dose of the biomarker is the tumor part higher in concentration of boron.

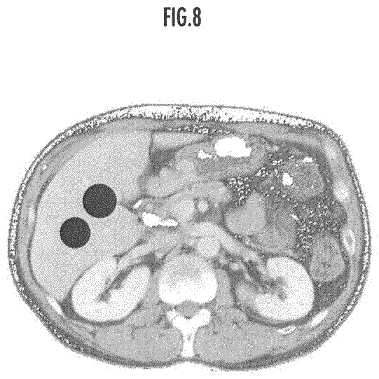

As shown in FIG. 8, the HOP 5 displays the dose distribution in the X-Y plane in the neutron irradiation range (prescribed range around Z=62 cm) on a display unit. In the dose distribution in the X-Y plane, portions where the treatment dose is less than a prescribed value (e.g., 1 GyE) (portions lower in absorbed dose of the biomarker) are displayed, for example, in blue color, and portions where the treatment dose is equal to or more than the prescribed value (1 GyE) (the tumor part higher in absorbed dose of the biomarker) are displayed, for example in red color (two black circles in FIG. 8).

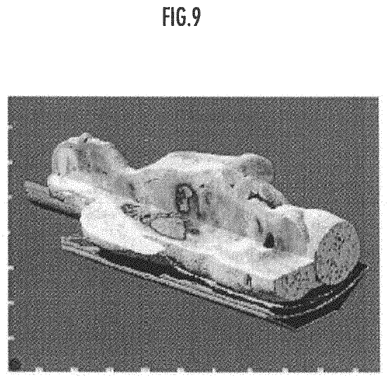

As shown in FIG. 9, the HOP 5 displays the dose distribution in the X-Y plane superimposed on contour information (D3 data) on the patient PA. The HOP 5 comprises a generation unit which generates contour information (3D data) on the patient PA based on the planar image (DICOM) at the time of biomarker administration, the CT image, and the MRI image.

The administrator of the HOP 5, the doctor in charge of the patient PA, or the like confirms the validity of the neutron irradiation sequence as a treatment plan by reviewing the dose distribution in the X-Y in the neutron irradiation range (prescribed range around Z=62 cm) and data that is the dose distribution in the X-Y plane superimposed on the contour information (3D data) on the subject to be treated.

For example, in the case where the treatment dose is equal to or more than the prescribed value (1 GyE) in normal tissues which are not in the tumor part, the neutron irradiation sequence is determined to be invalid. In the case where the treatment dose is equal to or more than the prescribed value (1 GyE) only in the tumor part, the neutron irradiation sequence is determined to be valid. It is also possible to determine that the neutron irradiation sequence is valid in the case where the treatment dose is equal to or more than the prescribed value (1 GyE) in normal tissues which are not in the tumor part, and to determine that the neutron irradiation sequence is invalid in the case where the treatment dose is equal to or more than the prescribed value (1 GyE) only in the tumor part.

When the neutron irradiation sequence as a treatment plan is determined to be valid, the HOP 5 transmits, as the irradiation sequence, the center of the neutron radiation range of the patient PA on the Z axis (Z=62 cm) and the mode for controlling the neutron irradiation of the first to sixth neutron irradiation devices 3A to 3F to the hospital where the first to sixth neutron irradiation devices 3A to 3F are installed. Each irradiation sequence information is stored in the management unit 7.

The irradiation sequence includes information on a total dose (boron dose+gamma dose+hydrogen dose+other doses), a specific treatment protocol of the irradiation sequence (treatment plan), operation programs of the first to sixth neutron irradiation devices 3A to 3F, and an operation program of the mobile table 17.

The controller 4 installed in the hospital operates the first to sixth neutron irradiation devices 3A to 3F and the mobile table 17, and performs neutron irradiation on the patient PA based on the received radiation sequence. At the time, the neutron irradiation along the irradiation sequence can easily be performed by loading and operating the operation programs of the first to sixth neutron irradiation devices 3A to 3F and the operation program of the mobile table 17, which are included in the irradiation sequence.

In the BNCT, when the patient PA is administered BPA (boron drug), formed by combining boron with a drug having a characteristic of staying in the tumor part irradiated with neutrons, by intravenous drip or the like, the tumor part of the patient PA incorporates the administered the BPA. When the tumor part incorporating the BPA is irradiated with neutrons (thermal neutrons), radiation (for example, alpha rays, ⁷Li, etc.) is generated in the tumor due to nuclear reaction between boron and neutrons. The generated radiation damages the tumor part. As a result, the BNCT destroys the tumor part with high selectivity.

Although the preferred embodiment of the present invention has been described in the foregoing, the present invention is not limited to the embodiment disclosed, and appropriate changes are possible without departing from the scope of the present invention for easy understanding of those skilled in the art.

For example, in the above embodiment, the HSP 6 and the management unit 7 are added as the BNCT treatment system 2. However, these units may not be included in the system.

In the above embodiment, six neutron irradiation devices are provided. However, the number of the neutron irradiation devices may be two to five, or seven or more, as long as the number is two or more.

### REFERENCE SIGNS LIST

2 . . . BNCT TREATMENT SYSTEM, 3A TO 3F . . . FIRST TO SIXTH NEUTRON IRRADIATION DEVICES, 4 . . . CONTROL DEVICE, 5 . . . HOP (NEUTRON IRRADIATION CONTROL FORMULATION UNIT) (VERIFICATION DEVICE), 6 . . . HSP (MONITORING DEVICE), 7 . . . MANAGEMENT UNIT

1. A BNCT treatment system which performs neutron capture therapy using a plurality of neutron irradiation devices which emit neutrons, the BNCT treatment system comprising:
a neutron irradiation control unit configured to control neutron irradiation by the neutron irradiation devices; and
a neutron irradiation control formulation unit configured to formulate a mode for controlling the neutron irradiation of the neutron irradiation devices by the neutron irradiation control unit, based on diagnostic data on a subject to be treated, wherein
the diagnostic data on the subject to be treated includes data on systemic distribution of absorbed dose when a biomarker is administered to the subject to be treated,
the neutron irradiation control formulation unit formulates the mode for controlling the neutron irradiation of the plurality of neutron irradiation devices by the neutron irradiation control unit through processes of
acquiring data on systemic distribution of absorbed dose when the biomarker is administered to the subject to be treated,
determining a portion higher in absorbed dose as a neutron irradiation range based on the data on the systemic distribution of the subject to be treated,
identifying a tumor part in the neutron radiation range, and
acquiring, based on size information on the identified tumor part, a mode for controlling the neutron irradiation of the plurality of neutron irradiation devices which has been executed in a case having similar size information on the tumor part.

2. The BNCT treatment system according to claim 1, comprising:
a verification device configured to
calculate treatment dose distribution obtained in a case where the neutron irradiation devices are controlled based on the mode for controlling the neutron irradiation formulated by the neutron irradiation control formulation unit, and
verify the mode for controlling the neutron irradiation formulated based on the calculated treatment dose distribution.

3. The BNCT treatment system according to claim 1, comprising:
a monitoring device configured to monitor the neutron irradiation devices.

4. The BNCT treatment system according to claim 2, comprising:
a monitoring device configured to monitor the neutron irradiation devices.
